(19) United States
(12) Patent Application Publication
(10) Pub. No.: US 2026/0314937 A1
(43) Pub. Date: Oct. 15, 2026

(54) WEARABLE TEMPERATURE SENSOR

(71) Applicant: Murata Vios, Inc., Woodbury, MN (US)

(72) Inventors: Atsuhiko HIRATA, Woodbury, MN (US); Nobuhiro KONDO, Woodbury, MN (US); John SAMPSON, Woodbury, MN (US); Rachel MYERS, Woodbury, MN (US); Yoshitaka HANE, Nagaokakyo-shi (JP); Yoshio YAMANAKA, Nagaokakyo-shi (JP)

(21) Appl. No.: 19/466,961

(22) Filed: Feb. 2, 2026

Related U.S. Application Data
(60) Provisional application No. 63/753,480, filed on Feb. 4, 2025.

(51) Int. Cl. A61B 5/01; A61B 5/00

(52) U.S. Cl. CPC A61B 5/01; A61B 5/6823; A61B 2562/0271

(57) ABSTRACT

A wearable sensor device includes a housing including an opening that extends between an exterior and an interior of the housing, a substrate in the interior of the housing, a window adjacent to the opening, and an infrared (IR) temperature sensor on a first principal surface of the substrate such that the IR temperature sensor receives radiation through the window.

### CROSS REFERENCE TO RELATED APPLICATIONS

This application claims the benefit of U.S. Patent Application No. 63/753,480 filed on Feb. 4, 2025. The entire contents of this application are hereby incorporated by reference.

### BACKGROUND OF THE INVENTION

### 1. Field of the Invention

The present invention relates to temperature sensors. More specifically, the present invention relates to wearable sensor devices that include infrared (IR) temperature sensors.

### 2. Description of the Related Art

It is known to use thermometers or temperature sensors to measure a patient's temperature. Known thermometers can use an IR temperature sensor to measure a patient's temperature. FIGS. 9 and 10 show known hand-held thermometers 300, 350 that include IR temperature sensors 310, 360 that can be used to measure the temperature of a patient's forehead. Similar hand-held thermometers can measure the temperature of a patient's ear canal. These known hand-help thermometers, including hand-held thermometers 300, 400, directly receive IR radiation from the patient's skin.

It is known to use temperature sensors with non-contact IR sensing. The non-contact, IR temperature sensors collect IR radiation from the IR temperature sensor's field of view (FOV) and calculate object temperature as well as ambient temperature using stored calibration data, which can be stored in read-only memory (ROM).

Known IR temperature sensors can use thermopile technology. These known thermopile IR temperature sensors include an enclosure made of a material with good, but not perfect, IR transparency, such as silicon. To ensure a proper temperature reading, calibration data of the thermopile IR temperature sensor can be stored in, for example, ROM, and an additional temperature sensor can be used that measures the temperature of the thermopile IR temperature sensor. The calibration data and the temperature of the thermopile IR temperature sensor can be used to calculate object temperature and ambient temperature.

It can be desirable to continuously or periodically measure a patient's skin temperature by using a wearable device to detect sooner a change in a patient's vital condition, as opposed to known manual temperature spot checks by a caregiver, for example, using the hand-help thermometers 300, 400 of FIGS. 9 and 10, which can also be a burden for the caregiver.

U.S. Patent Application Publication No. 2022/0031246 A1 discloses a wearable device that provides skin-temperature monitoring in which a digital temperature sensor near the electrocardiogram (ECG) circuit in the wearable device measures the patient's skin temperature. This known wearable device measures a patient's skin temperature using a temperature sensor embedded in a contact probe. But, in this case, it is difficult to achieve good measurement accuracy with such wearable devices because it is hard for the temperature sensor in the contact probe to maintain firm contact on the patient's skin.

U.S. Patent Application Publication No. 2023/0346309 A1 discloses a wearable device that monitors a patient's vital signs, including monitoring a patient's cardiac ECG, by being attached to the patient's skin using a patch electrode that includes a foam tape and/or sticky gel. While the electrically conductive patch electrode can provide electrical signals related to a patient's vital signs, the patch electrode is a poor thermal conductor, making it difficult to measure a patient's temperature.

A challenge of wearable devices that monitor a patient's cardiac ECG is that the wearable device should be defibrillation proof, meaning that the wearable device, when attached to a patient's skin, will not be damaged, will continue to operate, and will not harm the patient during defibrillation in which a high voltage is applied to a patient. The wearable device should also be watertight or waterproof to ensure that the wearable device is not damaged if a liquid comes into contact with the wearable device.

According to the standard IEC60601-1 (Edition 3.2 2020-08 Medical electrical equipment—Part 1: General requirements for basic safety and essential performance/8.9.1.15 CREEPAGE DISTANCES and AIR CLEARANCES for DEFIBRILLATION-PROOF APPLIED PARTS), at least a 4-mm creepage distance is required from the surface of a device to any electrical conductor within the wearable device to defibrillation proof the device. This 4-mm-creepage-distance requirement makes it difficult to design thin, small wearable devices if the electrically conductive IR temperature sensor within the wearable device is mounted on a surface of the wearable device. In addition, because silicon is not a good insulator, it is difficult to design a thin, small wearable device that uses IR temperature sensors with thermopile technology that include a silicon enclosure.

In addition, it can be desirable for the IR temperature sensor to not be accessible by the patient or other user (e.g., caregiver).

### SUMMARY OF THE INVENTION

To overcome the problems described above, example embodiments of the present invention provide wearable sensor devices that accurately detect a patient's skin temperature using an IR temperature sensor, while also being defibrillation proof and either watertight or waterproof.

According to an example embodiment of the present invention, a wearable sensor device includes a housing including an opening that extends between an exterior and an interior of the housing, a substrate in the interior of the housing, a window adjacent to the opening, and an infrared (IR) temperature sensor on a first principal surface of the substrate such that the IR temperature sensor receives radiation through the window.

The housing can enclose the IR temperature sensor and the substrate, and the IR temperature sensor can receive radiation emitted from a surface of a patient's skin via the window, when the wearable sensor device is attached to the patient's skin. The window can include polyolefin. The wearable sensor device can further include a window temperature sensor that measures a temperature of the window. The wearable sensor device can further include an additional temperature sensor to measure a temperature of the IR temperature sensor. The window can be in close contact with the IR temperature sensor.

The wearable sensor device can further include a thermally conductive plate between the window and the substrate and surrounding the IR temperature sensor. The thermally conductive plate can include a metal, and the thermally conductive plate and the IR temperature sensor can be soldered to the substrate. The thermally conductive plate can include a metal, and the thermally conductive plate and the IR temperature sensor can be mounted on the substate with thermal conductive paste. The substrate can be pressed towards the window with the thermally conductive plate in between the substrate and the window. The wearable sensor device can further include a buffer material that is on a second principal surface of the substrate opposite to the first principal surface and that presses against the substrate. The substrate can include a copper pattern connecting the IR temperature sensor and the thermally conductive plate. The substrate can include multiple copper layers connecting the IR temperature sensor and the thermally conductive plate. The substrate can include an alumina ceramic.

The wearable sensor device can further include a main substrate and a flexible printed circuit connected between the substrate and the main substrate. The window can be on an inner surface of the housing. The wearable sensor device can be configured to determine an object temperature using the IR temperature sensor, determine an ambient temperature based on either a temperature of the window or a temperature of the IR temperature sensor, and estimate a true object temperature based on the object temperature and the ambient temperature. The wearable sensor device can be configured to determine an object temperature using the IR temperature sensor, determine an ambient temperature based on either a temperature of the window or a temperature of the IR temperature sensor, and communicate the object temperature and the ambient temperature to a system that estimates a true object temperature based on the object temperature and the ambient temperature. The opening can include a chamfer that is wider toward the exterior of the housing. The wearable sensor device can further include at least one snap socket that engages an electrode connected to the patient's skin, when the wearable sensor device is attached to the patient's skin.

According to an example embodiment of the present invention, a wearable sensor device includes a housing including an opening that extends between an exterior and an interior of the housing, a substrate in the interior of the housing, and an infrared (IR) temperature on a first principal surface of the substrate and embedded in an insulating compound such that the IR temperature sensor receives radiation through the window.

The above and other features, elements, characteristics, steps, and advantages of the present invention will become more apparent from the following detailed description of example embodiments of the present invention with reference to the attached drawings.

### BRIEF DESCRIPTION OF THE DRAWINGS

FIGS. 1 and 2 show a wearable device that includes an infrared temperature sensor.

FIG. 3 shows the wearable device of FIG. 1 with a portion of the housing removed.

FIG. 4 shows the interior of the bottom housing of the wearable device of FIG. 1.

FIGS. 5 and 6 show cross-sectional views of two infrared temperature sensors that can be located adjacent to a window in the wearable device of FIG. 1.

FIG. 7 shows a cross-sectional view of another infrared temperature sensor that can be embedded in an insulating compound within the wearable device of FIG. 1.

FIG. 8 shows a cross-sectional view of the bottom housing of the wearable device of FIG. 1.

FIGS. 9 and 10 show known temperature sensors that use exposed IR temperature sensors to measure the temperature of a patient's skin.

FIG. 11 shows a bottom housing of a wearable device of the related art.

FIG. 12 shows a sectional view of a housing of the related art with a plastic exterior and a metal underlayer that includes a wide field of view.

FIG. 13 shows an IR temperature sensor with a metal can package that that be used in the plastic housing of FIG. 12.

FIG. 14 shows the field of view (FOV) of the IR temperature sensor of FIG. 13.

FIG. 15 shows a sectional view of a housing of the related art with a plastic exterior and a metal underlayer with a narrower FOV.

FIG. 16 shows a sectional view of a metal housing of the related art with a narrower FOV.

### DETAILED DESCRIPTION OF EXAMPLE EMBODIMENTS

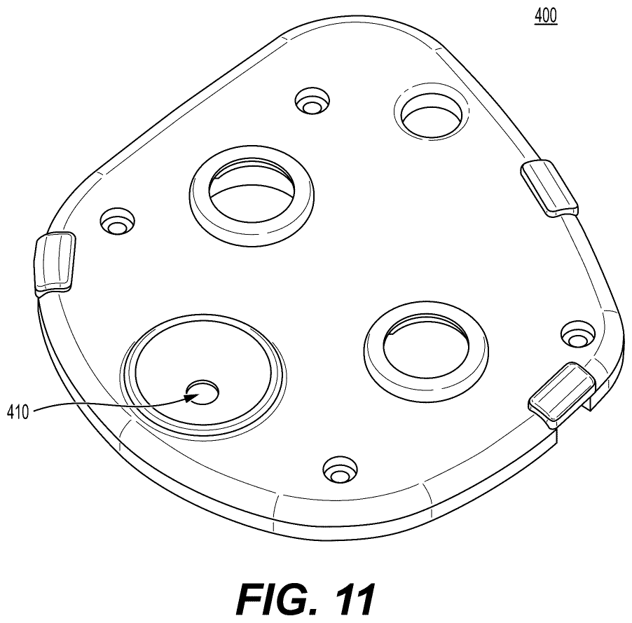

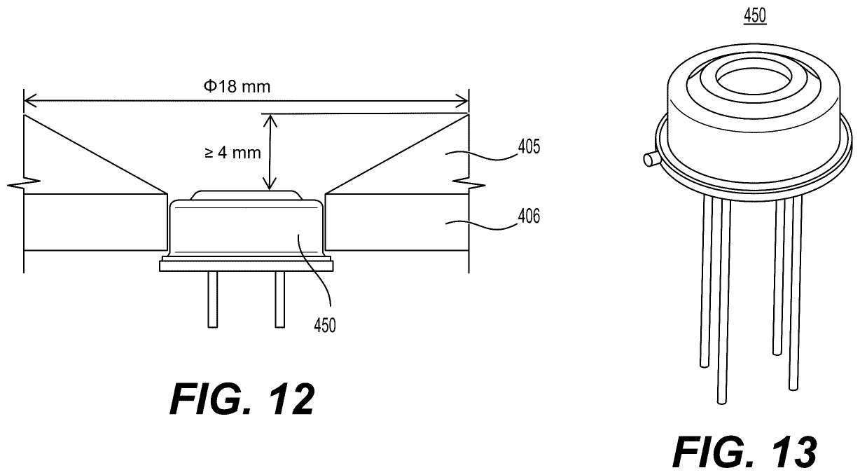

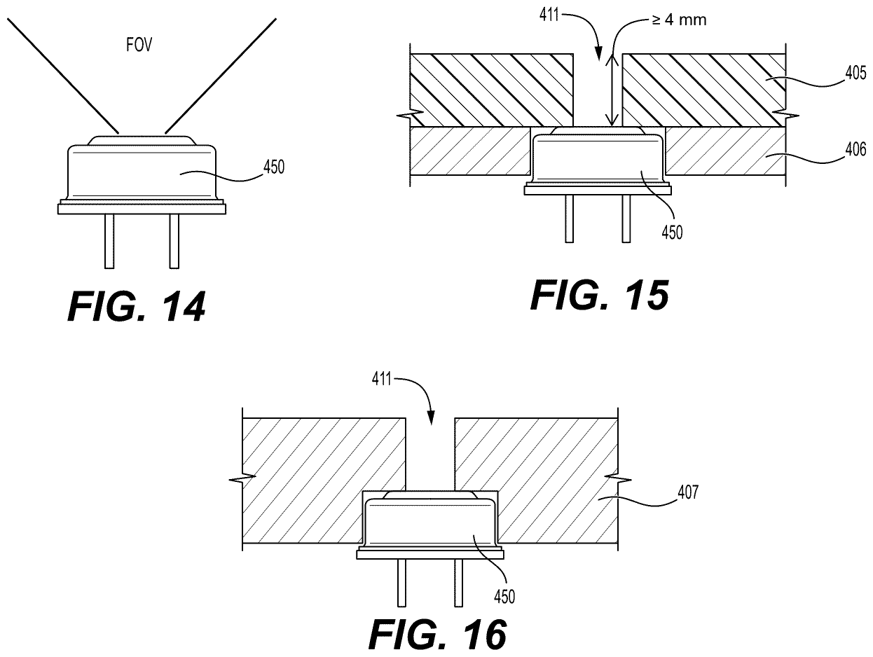

FIG. 11 shows a bottom housing 400 that can be used with a wearable sensor device, including, for example, the wearable device of U.S. Patent Application Publication No. 2023/0346309 A1, which is incorporated herein in its entirety. The bottom housing 400 includes a countersink 410 in which an IR temperature sensor can be located to receive IR radiation from a patient's skin. As shown in FIG. 12, an IR temperature sensor 450 can be located at the bottom of the countersink 410. As shown in FIG. 13, the IR temperature sensor 450 can include a metal can package, which is electrically conductive. FIG. 14 shows the field of view (FOV) of the IR temperature sensor 450 in which the IR temperature sensor 450 can receive IR radiation. The bottom housing 400 can include a plastic exterior 405 and a metal underlayer 406. The metal underlayer 406 helps maintain the IR temperature sensor 450 at a stable temperature. The countersink has an aperture size of 18 mm with a depth of 4 mm. Although the arrangement in FIG. 12 has an air clearance of 4 mm between the conductive IR temperature sensor 450 and the exterior surface of the plastic exterior 405, it can be undesirable that the IR temperature sensor 450 can be accessed or touched by a patient or caregiver.

FIGS. 15 and 16 show arrangements in which the countersink 410 is replaced with cylindrical through hole 411 that has 4-mm depth to provide an adequate creepage distance and air clearance between the exterior surface of the device and the electrically conductive IR temperature sensor 450. In FIG. 15, the bottom housing 400 includes a plastic exterior 405 and a metal underlayer 406, and in FIG. 16, the bottom housing does not include a plastic exterior and instead only has a metal exterior 407. The device in FIG. 15 provides defibrillation proofing by providing a 4-mm creepage distance and by not allowing a patient or a caregiver to access or touch the IR temperature sensor 450, but the accuracy of skin-temperature measurement is poor because the through hole 411 partially blocks the FOV of the IR temperature sensors 450, reducing the amount of IR radiation emitted from the patient's skin that is received by the IR temperature sensor 450. Additionally, the IR radiation emitted from the wall of the through hole 411 enters the IR temperature sensor 450, which affects the amount of IR radiation measured. In addition, the deep cylindrical through hole 411 requires cleaning if any dirt or debris gets in the cylindrical through hole 411, which can be burdensome to the patient or caregiver.

In the devices of FIGS. 15 and 16, if the temperature of the bottom housing 400 is known, a compensation calculation to cancel the effect of IR radiation emitted from the bottom housing 400 might be possible. For example, the IR temperature sensor 450 might include a temperature sensor to detect the temperature of the IR temperature sensor itself as the ambient temperature.

In the device in FIG. 15 with the plastic exterior 405, the temperature of the plastic exterior 405 can be different from the ambient temperature, which means that the temperature of the plastic exterior 405 must be measured or estimated. But it is difficult to accurately measure or estimate the temperature of the plastic exterior 405 because of the thickness of the plastic exterior 405 (i.e., at least 4 mm). Because the amount of IR radiation from the plastic exterior 405 is unknown, it is impossible to accurately measure the temperature.

The device in FIG. 16 includes a metal exterior 407. Because metal is a better thermal conductor than plastic and because of the use of metal exterior 407 instead of the plastic exterior 405, the temperature of IR temperature sensor 450 can be substituted for the temperature of the metal exterior 407. But, because the metal exterior 407 is electrically conductive, a 4-mm creepage distance is not provided, even though the depth of the through hole 411 is more than 4 mm, resulting in an arrangement that is not defibrillation proof.

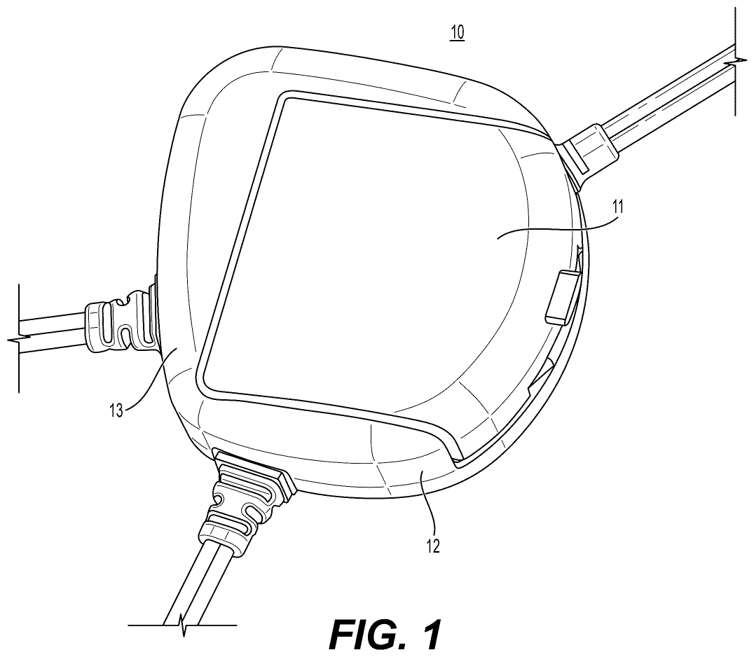

Specific example embodiments of a wearable device are shown in FIGS. 1-8 and are discussed below that accurately detect a patient's skin temperature using an IR temperature sensor, while also being defibrillation proof and either watertight or waterproof. But it should be understood that accurate temperature detection, while providing defibrillation proofness and either watertightness or waterproofness, can be provided with other wearable devices. Some example embodiments below provide a window in an opening through which IR radiation is transmitted to the IR temperature sensor. Other example embodiments use an IR temperature sensor that can withstand high voltages and that is embedded in an insulating compound.

FIGS. 1-8 show example embodiments of a wearable sensor device 10 that accurately measures a patient's skin temperature using an IR temperature sensor, while also being defibrillation proof and either watertight or waterproof.

In FIGS. 1-4, the wearable sensor device 10 includes two separable units: a battery pack 11 and a chest device 12. Although a separate battery pack 11 is shown in FIGS. 1-4, it is possible that the wearable sensor device 10 includes an integral battery pack that is not separable from the chest device 12. In FIGS. 1-4, the chest device 12 includes a two-part housing that is defined by a top housing 13 and a bottom housing 14. Instead of a two-part housing, it is also possible to use a unitary housing (e.g., a hinged clamshell housing) or any suitable multi-part housing. The housing of the wearable sensor device 10 can enclose an IR temperature sensor 31 (not shown in FIGS. 1-4) and a substrate on which the IR temperature sensor 31 (also not shown in FIGS. 1-4) is mounted. The wearable sensor device 10 allows the IR temperature sensor 31 to measure the emitted IR radiation from an object, for example, a patient's skin, without the surface of the IR temperature sensor being in direct contact with the object. The wearable sensor device 10 can measure the patient's skin temperature only or can include other vital data, including, for example, ECG, heart rate, respiration rate, SpO₂ levels, blood pressure, patient posture, and patient activity data.

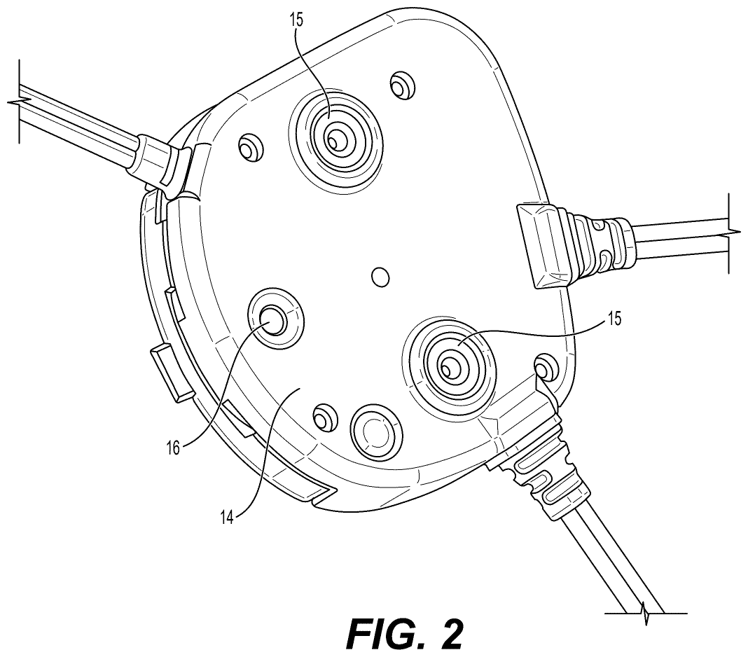

FIGS. 1 and 2 show that the wearable sensor device 10 includes three cables. But any number of cables can be used.

As shown in FIG. 2, the bottom housing 14 includes two snap sockets 15 and an opening 16. The snap sockets 15 can be snapped to electrodes on a patient's skin. Although FIG. 2 shows two snap sockets 15, any number of snap sockets 15 can be used. The distance between the center of the snap socket 15 and the center of the opening 16 can depend on the size of the electrodes that will be attached to the patient's skin and can be configured to ensure that the IR temperature sensor 31 (not shown in FIG. 2) has an adequate field of view (FOV). For example, the center of the snap socket 15 can be spaced away from the center of the opening 16 by at least 30 mm in plan view. As specific examples, the center of the snap socket 15 can be spaced away from a center of the opening 16 by about 34 mm or about 35 mm, within manufacturing and/or measurement tolerances. The opening 16 can extend between the exterior of the bottom housing 14 and the interior of the bottom housing 14. The opening 16 can be chamfered such that the opening 16 widens toward the exterior of the bottom housing 14 to ensure that the IR temperature sensor 31 (not shown in FIG. 2) has an adequate FOV. But it is possible that the opening 16 is not chamfered, for example, if the IR temperature sensor 31 has a narrow FOV and/or if the opening 16 is wide enough to accommodate the FOV of the IR temperature sensor 31.

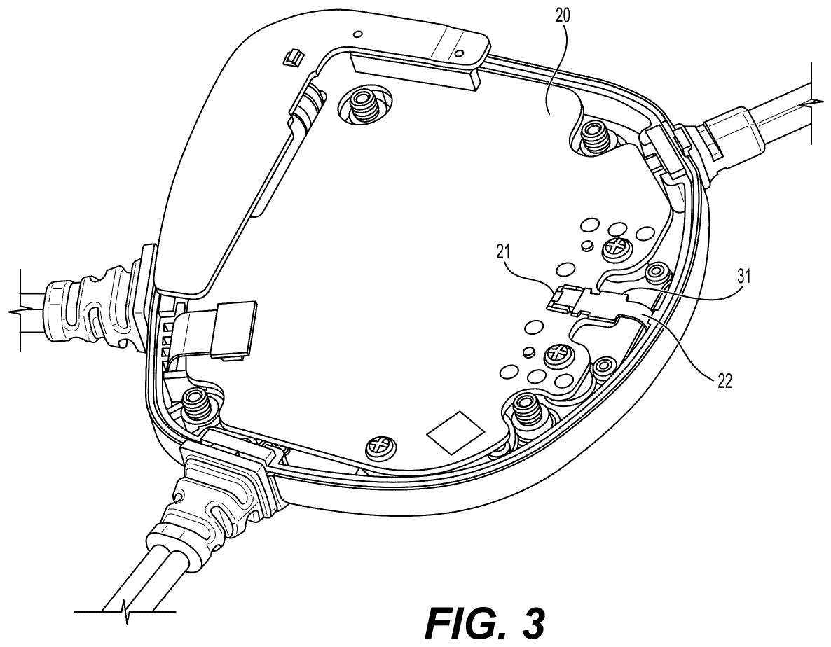

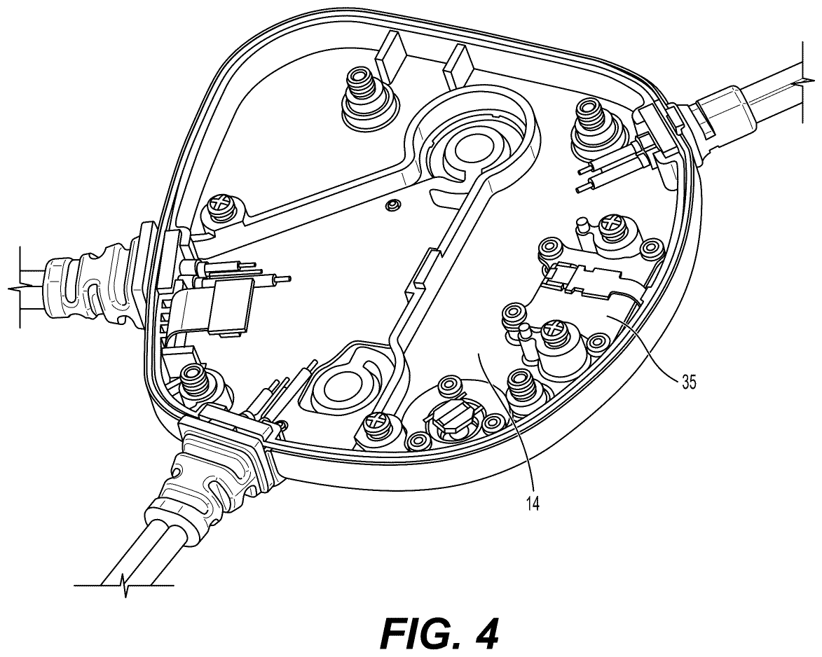

FIGS. 3 and 4 show the chest device 12 without the top housing 13 to show the interior of the housing of the wearable sensor device 10. The circuitry for providing the various functions of the wearable sensor device 10 can be included within the housing of the wearable sensor device 10. A main substrate 20 is included within the housing of the chest device 12. The main substrate 20 can include any suitable substrate, including, for example, a printed circuit board (PCB). For simplicity, the main substrate 20 does not include any components, but components can be included on the main substate 20. The components can be active or passive components and can provide the functions of the chest device 12, including, for example, monitoring of patient's vital data, such as, ECG, heart rate, respiration rate, SpO₂ levels, blood pressure, posture, and activity data. As shown in FIG. 3, it is possible for additional substrate(s) to be included within the housing of the chest device 12. For example, an additional substrate with communication circuitry can be provided such that the communication circuitry is not blocked by the battery pack 11 when the wearable sensor device 10 is attached to a patient. Any suitable communication circuitry can be provided, including, for example, WiFi circuitry as shown in FIG. 3, but also, for example, Bluetooth circuitry. In the example embodiments of FIGS. 1-8, the IR temperature sensor 31 is located on a separate substrate 30, but it is also possible for the IR temperature sensor 31 to be located on the main substrate 20. The IR temperature sensor 31 can be located on the separate substrate 30 to isolate the IR temperature sensor 31 from any heating emitting components on the main substrate 20, which can improve accuracy of temperature measurements. The substrate 30 can be any suitable substrate, including a PCB. In addition, the substrate 30 can include, for example, alumina ceramic or other suitable thermally conductive material.

The IR temperature sensor 31 is not shown in FIGS. 3 and 4 but is connected to the main substrate 20 by a flex tail 22 and a connector 21. The flex tail 22 can be defined by a flexible printed circuit. One end of the flex tail 22 can be connected to substrate 30 (not shown in FIGS. 3 and 4) on which the IR temperature sensor 31 is located, and the other end of the flex tail 22 can be connected to the connector 21. The connector 21 can mate with a corresponding connector on the main substrate 20. Instead of the using connector 21, it is also possible to directly attach the flex tail 22 to the main substrate 20.

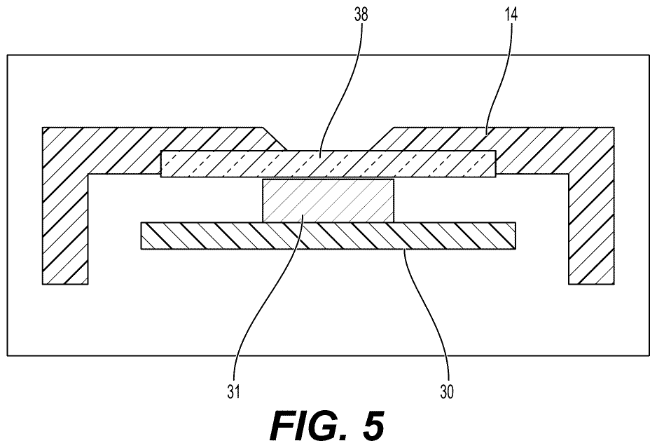

FIGS. 5-7 show example arrangements of the IR temperature sensor 31 within the housing of the wearable sensor device 10. The IR temperature sensor 31 is mounted on the substrate 30 adjacent to the opening 16 of the bottom housing 14 such that the IR temperature sensor 31 receives radiation, including IR radiation, through the opening 16. The opening 16 can be chamfered to ensure that the IR temperature sensor 31 has an adequate FOV to receive IR radiation emitted from an object (e.g., a patient's skin). But, as explained above, it is possible that the opening 16 is not chamfered.

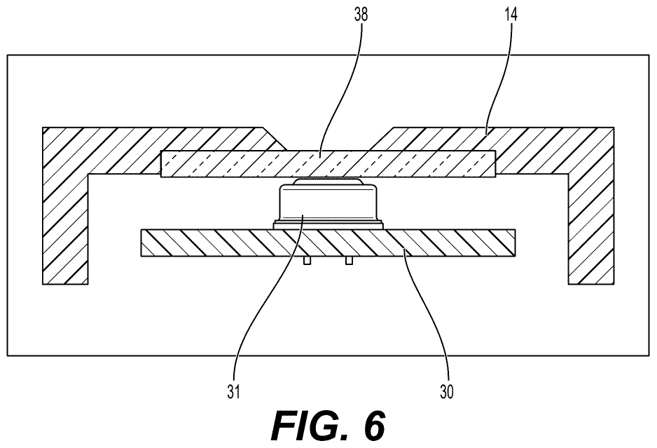

FIGS. 5 and 6 show a window 38 between the IR temperature sensor 31 and the opening 16. The window 38 does not compromise the defibrillation proofing or either the watertightness or the waterproofing of the wearable sensor device 10. The window 38 can cover the IR temperature sensor 31 so that IR temperature sensor 31 is not exposed to the exterior of the housing of the wearable sensor device 10, helping defibrillation proof the wearable sensor device 10. The window 38 can be attached to the housing of the wearable sensor device 10 such that the window 38 covers the opening 16 such that liquid is prevented from entering the housing of the wearable sensor device 10.

The window 38 can be in close contact with the IR temperature sensor 31, meaning that the window 38 is in direct contact with the IR temperature sensor 31, that the window 38 is adjacent to the IR temperature sensor 31 with a narrow air gap in between the window 38 and the IR temperature sensor 31, or that the window 38 is adjacent to the IR temperature sensor 31 with a narrow gap in between the window 38 and the IR temperature sensor 31 filled with a thin material. For example, the narrow gap can be about 0.2 mm, within manufacturing and/or measurement tolerances. The window 38 can be made of a material with sufficient IR transparency so that IR radiation can pass through the window 38 and can be received by the IR temperature sensor 31. When the wearable sensor device 10 is attached to a patient, IR radiation emitted from the patient's skin can pass through the window 38 and can be received by the IR temperature sensor 31. The window 38 can be made of any suitable material with sufficient IR radiation transmittance. For example, the window 38 can include polyolefin, silicon, silicon crystal, sapphire, or diamond. Polyolefin is a relatively good IR radiation transmitter that reduces or minimizes the impact the window 38 has on the accuracy of temperature measurements, and polyolefin is a good insulator that protects the IR temperature sensor 31 and other internal circuitry of the wearable sensor device 10 from high voltages applied to the exterior of the housing of the wearable sensor device 10. The wearable sensor device 10 can estimate the temperature of the patient's skin based, in part, on the IR radiation emitted from the patient's skin and received by the IR temperature sensor 31. FIG. 5 shows an example of an IR temperature sensor 31 that can be surface mounted to the substrate 30, and FIG. 6 shows an example of an IR temperate sensor 31 that includes pins that can be through-hole mounted to the substrate 30. It should be understood that FIGS. 5 and 6 show examples of the types of IR temperature sensors that can be used and that it is possible to use other types of IR temperature sensors. The IR temperature sensor 31 can be attached to the substrate 30 with solder, thermal conductive paste, or other similar material that facilitates thermal conduction between the IR temperature sensor 31 and the substrate 30.

The wearable sensor device 10 can include a window temperature sensor that measures the temperature of the window 38. If the window temperature sensor is a contact sensor in contact with the window 38, then the window temperature sensor should be positioned not to block the FOV of the IR temperature sensor 31. Alternative to or in addition to the window temperature sensor, the IR temperature sensor 31 can include an additional internal temperature sensor that measures the temperature of the IR temperature sensor 31 itself, which the IR temperature sensor 31 can output as the ambient temperature. The additional internal temperature sensor can be any suitable temperature sensor, including, for example, a thermistor or a thermopile. The patient's skin temperature can be estimated using the received IR radiation (object temperature) and either the temperature of the window 38 or the temperature of the IR temperature sensor 31 (ambient temperature).

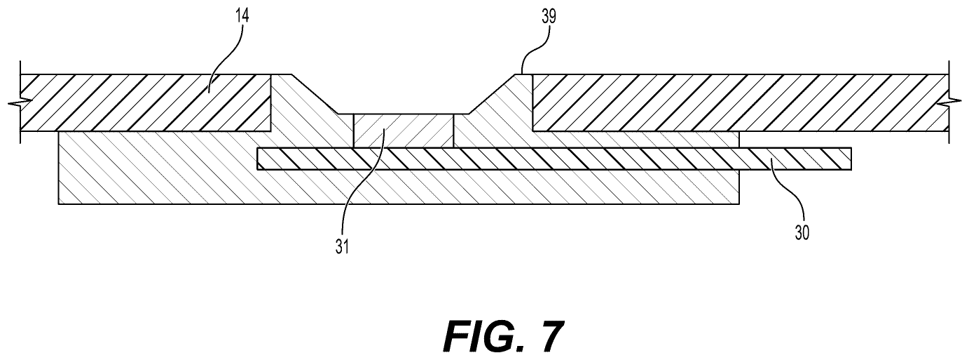

In FIG. 7, instead of using a window 38, the IR temperature sensor 31 is embedded in an insulating compound 39 such that a surface of the IR temperature sensor 31 is exposed to receive IR radiation. As shown in FIG. 7, both the IR temperature sensor 31 and a portion of the substrate 30 can be embedded in the insulating compound 39. To provide defibrillation proofing, the exposed surface of the IR temperature sensor 31 needs to be able withstand high voltages. In FIG. 7, there is no window to complicate determining an object's temperature, as discussed below. That is, an object's temperature can be determined by measuring the IR radiation emitted by the object.

In the example embodiments of FIGS. 5 and 6, if the window 38 has an ideal 100% IR radiation transmittance, then the window 38 would not affect accuracy of the temperature measurement. But in reality, even if a good IR transparent material is chosen for the window 38, then a certain amount of IR radiation is reflected or absorbed by the window 38, and the window 38 emits a certain amount of IR radiation, depending on the temperature of the window 38 itself. If the window 38 is warm, the more IR radiation will be emitted by the window and received by the IR temperature sensor 31. And if the window 38 is cold, then the less IR radiation will be emitted by the window 38 and the less IR radiation from the window 38 will be received by the IR temperature sensor 31. Thus, accurate temperature measurements can be difficult because of the existence of the window 38.

For example, if the wearable sensor device 10 is attached to a patient's chest and if the patient is covered with a blanket such that the window 38 is warmed by the patient's body, then the IR temperature sensor 31 can receive more IR radiation from the window 38, which could lead to a skin temperature measurement that is higher than the actual skin temperature.

For example, if the window 38 is not warmed up because the wearable sensor device 10 is recently attached to a patient's chest or because air is blown between the wearable sensor device 10 and the patient's skin, then the IR temperature sensor 31 receives less IR radiation from the window 38, which could lead to a skin temperature measurement that is lower than the actual skin temperature.

The temperature of the window 38 can be measured, either directly through an additional temperature sensor that measures the temperature of the window 38 or indirectly through an internal temperature sensor in the IR temperature sensor 31 that measures the temperature of the IR temperature sensor 31 itself, with the assumption that the temperature of the IR temperature sensor 31 is the same or about the same temperature as the window 38.

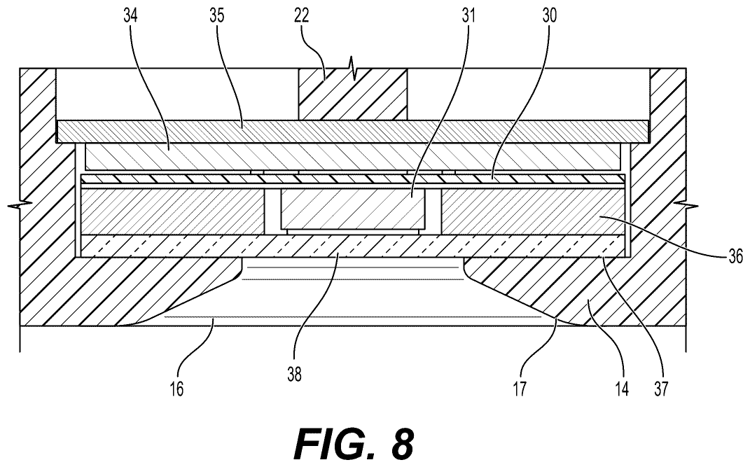

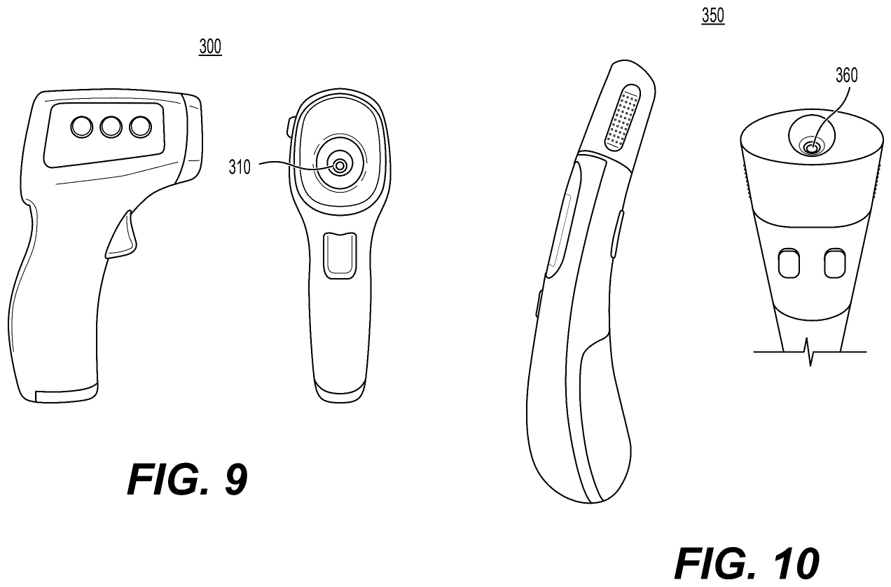

FIG. 8 shows an example structure of the IR temperature sensor 31 within the housing of the wearable sensor device 10. As discussed above, the IR temperature sensor 31 can be located on a substrate 30, and the window 38 can be between the opening 16 in the housing of the wearable sensor device 10 and the IR temperature sensor 31. The window 38 can be attached to an interior surface of the housing of the wearable sensor device 10 with double-sided tape 37 or any other suitable manner. Attaching the window 38 to the interior of the housing of the wearable sensor device 10 allows a compressive force to be applied to the window 38, which can ensure good thermal contact between the window 38 and the components being pressed against the window 38 (e.g., the IR temperature sensor 31 and the thermally conductive plate 36 as discussed below).

A thermally conductive plate 36 can be located between the substrate 30 and the window 38 and can surround the IR temperature sensor 31. The thermally conductive plate 36 is optional and is not necessary in all applications. The thermally conductive plate 36 can include any suitable thermal conductor, including, for example, a metal (such as aluminum or copper), silicon, diamond, silicon crystal, sapphire, etc. The thermally conductive plate 36 does not have to be connected to the substrate 30. If the thermally conductive plate 36 is attached to the substrate 30, the thermally conductive plate 36 can be attached to the substrate 30 in any suitable manner, including, for example, using solder or thermal conductive paste.

The thermally conductive plate 36 can be in thermal contact with both the IR temperature sensor 31 and the window 38 so that the temperature of the window 38 and the temperature of the IR temperature sensor 31 are at the same or about the same temperature. For example, the temperatures of the window 38 and the IR temperature sensor 31 can be less than about 0.1 °C, within measurement tolerances. But in some applications, the temperature difference between the window 38 and the IR temperature sensor 31 can be larger. By ensuring that the temperature of the window 38 and the temperature of the IR temperature sensor 31 are the same or about the same temperature, the internal temperature sensor of the IR temperature sensor 31 that measures the temperature of the IR temperature sensor 31 can be used to estimate the temperature of the window 38. If the wearable sensor device 10 includes an additional temperature sensor that measures the temperature of the window, then it is not necessary to use the internal temperature sensor of the IR temperature sensor 31 to estimate the temperature of the window 38, in which case the thermally conductive plate 36 might not be necessary.

A buffer material 34 can be located between a retainer plate 35 and the substrate 30 on a side of the substrate 30 opposite to the IR temperature sensor 31. The buffer material 34 can be in pressure contact with the substrate 30 to ensure sufficient thermal contact between the thermally conductive plate 36 and the window 38. Ensuring sufficient thermal contact can improve accuracy of temperature measurements.

The substrate 30 can include one or more metal patterns and/or one or more metal layers that provide one or more thermally conductive paths between components attached to the substrate 30, including thermally conductive path(s) between the IR temperature sensor 31 and thermally conductive plate 36, which can improve the accuracy of the temperature measurement. The one or more metal patterns and/or one or more metal layers can include copper, gold, nickel, etc. For example, if the substrate 30 includes alumina ceramic, then the substrate 30 can include gold or nickel. If the substrate 30 is a double-sided copper-clad substrate or a multilayer substrate, then multiple layers of copper patterns can be stacked on top of each other, including the conductor layers on the top and bottom of the substrate 30 and/or within the substrate 30 (i.e., on an internal layer of the substrate 30).

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1. A wearable sensor device comprising:
a housing including an opening that extends between an exterior and an interior of the housing;
a substrate in the interior of the housing;
a window adjacent to the opening; and
an infrared (IR) temperature sensor on a first principal surface of the substrate such that the IR temperature sensor receives radiation through the window.

2. The wearable sensor device of claim 1, wherein
the housing encloses the IR temperature sensor and the substrate; and
the IR temperature sensor receives radiation emitted from a surface of a patient's skin via the window, when the wearable sensor device is attached to the patient's skin.

3. The wearable sensor device of claim 1, wherein the window includes polyolefin.

4. The wearable sensor device of claim 1, further comprising a window temperature sensor that measures a temperature of the window.

5. The wearable sensor device of claim 1, further comprising an additional temperature sensor to measure a temperature of the IR temperature sensor.

6. The wearable sensor device of claim 1, wherein the window is in close contact with the IR temperature sensor.

7. The wearable sensor device of claim 1, further comprising a thermally conductive plate between the window and the substrate and surrounding the IR temperature sensor.

8. The wearable sensor device of claim 7, wherein
the thermally conductive plate includes a metal; and
the thermally conductive plate and the IR temperature sensor are soldered to the substrate.

9. The wearable sensor device of claim 7, wherein
the thermally conductive plate includes a metal; and
the thermally conductive plate and the IR temperature sensor are mounted on the substate with thermal conductive paste.

10. The wearable sensor device of claim 7, the substrate is pressed towards the window with the thermally conductive plate in between the substrate and the window.

11. The wearable sensor device of claim 10, further comprising a buffer material that is on a second principal surface of the substrate opposite to the first principal surface and that presses against the substrate.

12. The wearable sensor device of claim 7, wherein the substrate includes a copper pattern connecting the IR temperature sensor and the thermally conductive plate.

13. The wearable sensor device of claim 7, wherein the substrate includes multiple copper layers connecting the IR temperature sensor and the thermally conductive plate.

14. The wearable sensor device of claim 7, wherein the substrate includes an alumina ceramic.

15. The wearable sensor device of claim 1, further comprising:
a main substrate; and
a flexible printed circuit connected between the substrate and the main substrate.

16. The wearable sensor device of claim 1, wherein the window is on an inner surface of the housing.

17. The wearable sensor device of claim 1, wherein the wearable sensor device is configured to:
determine an object temperature using the IR temperature sensor;
determine an ambient temperature based on either a temperature of the window or a temperature of the IR temperature sensor; and
estimate a true object temperature based on the object temperature and the ambient temperature.

18. The wearable sensor device of claim 1, wherein the wearable sensor device is configured to:
determine an object temperature using the IR temperature sensor;
determine an ambient temperature based on either a temperature of the window or a temperature of the IR temperature sensor; and
communicate the object temperature and the ambient temperature to a system that estimates a true object temperature based on the object temperature and the ambient temperature.

19. The wearable sensor device of claim 1, wherein the opening includes a chamfer that is wider toward the exterior of the housing.

20. The wearable sensor device of claim 1, further comprising at least one snap socket that engages an electrode connected to the patient's skin, when the wearable sensor device is attached to the patient's skin.

21. A wearable sensor device comprising:
a housing including an opening that extends between an exterior and an interior of the housing;
a substrate in the interior of the housing; and
an infrared (IR) temperature on a first principal surface of the substrate and embedded in an insulating compound such that the IR temperature sensor receives radiation through the window.
